(19) United States
(12) Patent Application Publication
(10) Pub. No.: US 2021/0230134 A1
(43) Pub. Date: Jul. 29, 2021

(54) VITAMIN E DERIVATIVES AND THEIR USE AS MULTI-SCALE IMAGING AGENTS

(71) Applicant: EXACT DELIVERY INC., Palmer Rapids (CA)

(72) Inventors: Drew MARQUARDT, LaSalle (CA); Mikel GHELFI, Trimbach (CH); Jeffrey ATKINSON, St. Catharines (CA); Thad A. HARROUN, St. Catharines (CA)

(73) Assignee: Exact Delivery Inc., Palmer Rapids, ON (CA)

(21) Appl. No.: 17/050,897

(22) PCT Filed: May 3, 2019
(86) PCT No.: PCT/CA2019/050585

§ 371 Date: Oct. 27, 2020

Related U.S. Application Data
(60) Provisional application No. 62/666,837, filed on May 4, 2018.

(51) Int. Cl. C07D 311/70; A61K 49/10; A61K 49/18; A61K 9/127; A61K 49/00

(52) U.S. Cl. CPC C07D 311/70; A61K 49/10; A61K 49/0052; A61K 9/1271; A61K 49/0084; A61K 49/1812

(57) ABSTRACT

The present disclosure relates to Vitamin E derivatives as multi-scale imaging agents. In particular, the present disclosure relates to isotopically labeled Vitamin E derivatives, and their use as multi-scale imaging agents.

### RELATED APPLICATIONS

This Application claims priority to U.S. Provisional Patent Application No. 62/666,837 filed May 4, 2018, the contents of which are incorporated by reference herein in their entirety.

### FIELD

The present disclosure relates to Vitamin E derivatives as multi-scale imaging agents. In particular, the present disclosure relates to isotopically labeled Vitamin E derivatives, and their use as multi-scale imaging agents.

### INTRODUCTION

The use of fluorescence microscopy to observe a labeled molecule in cells as it is absorbed and transported through different cellular compartments can be achieved with photostable fluorescent probes. In particular, probes with a fluorophore that absorbs at wavelengths greater than 500 nm makes for better light penetration in cells and tissues. Furthermore, the fluorophore should be photostable during the course of this irradiation.

Positron Emission Tomography (PET) uses radioactive probes to search the body for cancer, helping doctors better diagnose and track the course of disease treatment. One of the key elements of PET imaging agents is to be able to address the technical issues of optimized and expedient production of ¹⁸F since it has a short half-life of 109 minutes.

Magnetic Resonance Imaging (MRI) uses probes that have nuclei containing an odd number of protons and/or neutrons (nonzero nuclear spin, S≠0). Traditionally, hydrogen are the nuclei monitored with MRI. With new instruments and new techniques to increase MRI signal, the use of other S≠0 nuclei present new opportunities for site specific and targeted MRI agents. One of these novel technical approaches is the use of Dynamic Nuclear Polarization (DNP).

Dynamic Nuclear Polarization (DNP) couples the spin of an unpaired electron with the nuclear spin of a S≠0 nuclei. Through the process, a MRI signal can be increased by several orders of magnitude.

Lipid based imaging agent delivery technologies provide great opportunities in the medical imaging field. Liposomes allow for hydrophobic agents to be introduced in the body and circulated. Furthermore, lipid based carriers allow for the inclusion of hydrophobic stable radicals which can be used as the source of an unpaired electron for the use of DNP.

### SUMMARY

The present disclosure relates to Vitamin E derivatives, which are useful as multi-scale imaging agents. In one embodiment, the Vitamin E derivatives of the present disclosure are useful as imaging agents in fluorescence microscopy, and are simultaneously useful as PET, MRI and/or DNP enhanced MRI agents.

In one embodiment of the disclosure, the Vitamin E derivatives are compounds of the Formula (I)

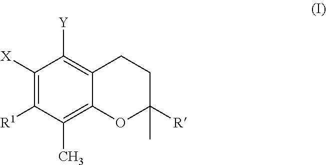

wherein
R′ is

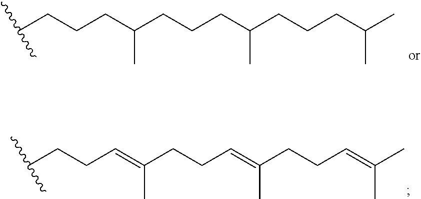 [SMILES: *CCCC(C)CCCC(C)CCCC(C)C ; *CC/C=C(\C)CC/C=C(\C)CCC=C(C)C]

R¹ is H or CH₃;
X is ¹⁸F, ¹⁹F or OH;
Y is ¹⁸F, ¹⁹F or CH₃;
wherein when X is ¹⁸F or ¹⁹F, Y is CH₃, and when Y is ¹⁸F or ¹⁹F, X is OH.

The present disclosure also includes compounds of the Formula (II) for use as multi-scale imaging agents,

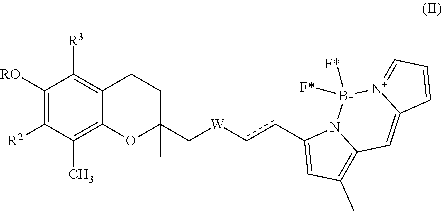

wherein
R² and R³ are independently or simultaneously H or CH₃; R is H, (C₁-C₆)-alkyl; —C(═O)(C₁-C₆)-alkyl; —C(═O)(C₁-C₆)-alkyl-COOH; or —CH₂—(C₁-C₆)-alkyl-COOH;
W is (C₂-C₄)-alkylene or (C₅-C₆)-heteroaryl;
F* is independently or simultaneously ¹⁸F or ¹⁹F, wherein at least one of F* is ¹⁸F; and [symbol] is a single bond or a double bond.

The present disclosure also includes facile syntheses for the preparation of the compounds of Formula (I).

The present disclosure also includes methods of diagnosing and/or monitoring a disease state in which α-tocopherol transfer protein is involved. For example, the disease stats include ataxias with vitamin E deficiency and Nonalcoholic Fatty Liver Disease (NASH) (see Hirsova; Thrasher; Erhardt; Hadi).

Other features and advantages of the present application will become apparent from the following detailed description. It should be understood, however, that the detailed description and the specific examples while indicating preferred embodiments of the application are given by way of illustration only, since various changes and modifications within the spirit and scope of the application will become apparent to those skilled in the art from this detailed description.

### BRIEF DESCRIPTION OF THE DRAWINGS

The present disclosure will now be described in greater detail with reference to the drawings in which:

FIG. 1 shows the fluorine exchange of thienyl-ene-BODIPY, followed by UV/Vis.

FIG. 2 are graphs showing the viability of cultured mouse cells in the presence of the compounds of the disclosure.

FIG. 3 are (A-C) graphs showing the viability of cultured mouse cells in the presence of compounds of the disclosure and liposomes containing compounds of the disclosure, and (D) an absorption and emission spectra for a compound of the disclosure.

FIG. 4 shows micrographs of the cellular uptake of a compound of the disclosure.

FIG. 5 is a graph showing fluorescence titrations on α-TTP of a compound of the disclosure.

FIG. 6 is a graph showing fluorescence intensity of competitive displacement of α-tocopherol transfer protein using a compound of the disclosure.

FIG. 7 is a graph showing fluorescence intensity in a titration of a compound of the disclosure with α-tocopherol transfer protein.

FIG. 8 shows (A) fluorescence microscopy of cells expressing α-tocopherol transfer protein loaded with a compound of the disclosure; and (B) a graph showing quantified fluorescence.

### DESCRIPTION OF VARIOUS EMBODIMENTS

### (I) Definitions

The term “multi-scale imaging agent” as used herein refers to a compound which is able to fluoresce under fluorescence microscopy and which are also isotopically labelled and are useful in PET, MRI and/or DNP enhanced MRI.

The term “heteroaryl”” as used herein means a monocyclic ring system containing 5 or 6 atoms, of which, unless otherwise specified, one, two, three, four or five are a heteromoiety independently selected from N, NH, NC₁₋₆ alkyl, O and S and includes thienyl, furyl, pyrrolyl, pyrididyl, indolyl, and the like.

The term “(C₁-C₆)-alkyl” as used herein means straight and/or branched chain, saturated alkyl radicals containing from one to six carbon atoms and includes methyl, ethyl, propyl, isopropyl, n-butyl, s-butyl, isobutyl, t-butyl, 2,2-dimethylbutyl, n-pentyl, 2-methylpentyl, 3-methylpentyl, 4-methylpentyl, n-hexyl and the like.

The suffix “ene” added on to any of the above groups means that the group is divalent, i.e. inserted between two other groups.

The term “halo” or “halogen” as used herein means halogen and includes chloro, fluoro, bromo and iodo.

The term “a” as used herein is intended to mean “one or more” or “at least one”, except where it is clear from the text that it means one.

### (II) Multi-Scale Imaging Agents

The present disclosure relates to isotopically labeled Vitamin E derivatives which are useful as multi-scale imaging agents, such as positron emission tomography, magnetic resonance imaging and microscopy. In one embodiment, the Vitamin E derivatives have a fluorophore which can fluoresce under light microscopy, and as well, the derivatives are isotopically labeled with radioactive fluorine and are therefore useful as agents in PET, single-photon emission tomography (SPECT), MRI and/or DNP enhanced MRI, and for visualizing biopsied cells.

In one embodiment of the disclosure, the Vitamin E derivatives are compounds of the Formula (I)

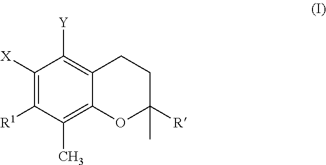

wherein
   R¹ is

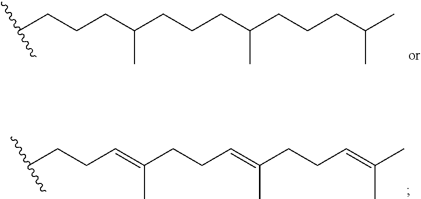 [SMILES: *CCCC(C)CCCC(C)CCCC(C)C ; *CC/C=C(\C)CC/C=C(\C)CCC=C(C)C]

R¹ is H or CH₃;
X is ¹⁸F, ¹⁹F or OH;
Y is ¹⁸F, ¹⁹F or CH₃;
wherein when X is ¹⁸F or ¹⁹F, Y is CH₃, and when Y is ¹⁸F or ¹⁹F, X is OH.

In one embodiment of the disclosure, R¹ is CH₃.

In one embodiment of the disclosure, the compound of the Formula (I) is

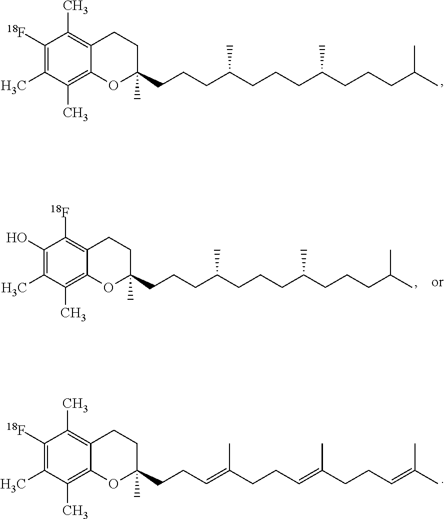

In one embodiment, when the compound of the Formula (I) contains an 18 F, the compound is useful for PET, and when the compound contains an 19F, the compound is useful for MRI.

### (III) Methods of Imaging Using Vitamin E Derivatives

In one embodiment, the present disclosure also includes compounds of the Formula (II) for use as multi-scale imaging agents,

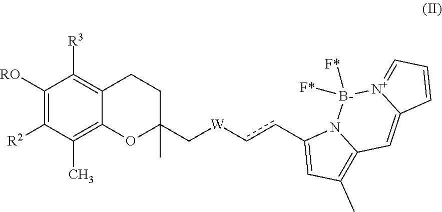

wherein
R² and R³ are independently or simultaneously H or CH₃;
R is H, (C₁-C₆)-alkyl, —C(═O)(C₁-C₆)-alkyl, —C(═O)(C₁-C₆)-alkyl-COOH, or —CH₂—(C₁-C₆)-alkyl-COOH;
W is (C₂-C₄)-alkylene, phenyl or (C₅-C₆)-heteroaryl;
each F* is independently or simultaneously ¹⁸F or ¹⁹F, wherein at least one of F* is ¹⁸F; and
[symbol] is a single bond or a double bond

In another embodiment, R² and R³ are CH₃.

In another embodiment, R is H.

In another embodiment, W is

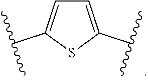 [SMILES: *c1ccc(*)s1]

In another embodiment, the compound of Formula (II) is

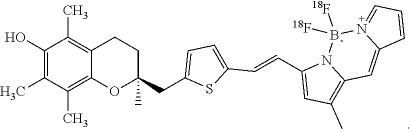

In another embodiment, the compound of the Formula (II) is

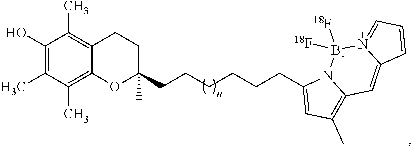

wherein n is an integer between 1-3.

In an embodiment of the disclosure, the compounds of the Formula (I) and (II) bind to α-TTP (α-tocopherol transfer protein) and are taken up with specificity by the liver, where α-TTP is predominantly expressed. In one embodiment, the multi-scale imaging of an organ and/or tissue, such as the liver, is useful for the diagnosis and subsequent treatment of non-alcoholic fatty liver (NAFL) and non-alcoholic steatohepatitis (NASH), pathologies common in obese and diabetic patients.

In another embodiment, the compounds of the Formula (I) and (II) are useful for imaging a subject using PET or MRI. In another embodiment, the compounds of the Formula (I) and (II) are useful for imaging a disease or condition in a subject using PET or MRI. In another embodiment, the disease or condition is a neurological disease such as Alzheimer's disease or Parkinson's disease, cancer such as breast cancer, or heart disease and/or other circulatory issues.

The present disclosure also includes methods of diagnosing and/or monitoring a disease state in which α-TTP is involved. In one embodiment, the present disclosure includes a method of diagnosing and/or monitoring a disease state in which α-TTP is involved, the method comprising the steps of:
a. administering to a patient being evaluated for the disease state an effective amount of a compound of the Formula (I) or (II)

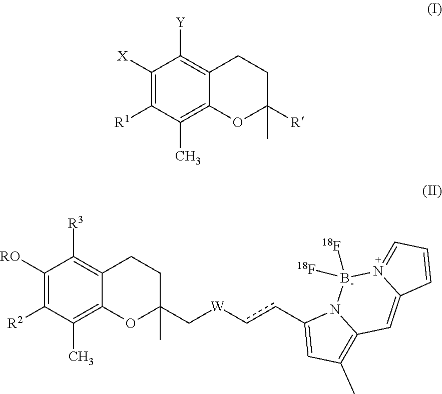

wherein R, R′, R¹, R², R³, X, Y, W and F* are as defined above,
b. allowing sufficient time for the compound of Formula (I) or (II) to bind to α-TTP; and
c. diagnosing and/or monitoring the disease state using light fluorescence microscopy and/or positron emission tomography.

In one embodiment, the disease state is non-alcoholic fatty liver (NAFL) and non-alcoholic steatohepatitis (NASH).

In another embodiment, the compounds of the Formula (I) and (II) are for formulated as or within liposomes for delivery of the agents to the subject. In one embodiment, the compounds of the Formula (I) and/or (II) are formulated with a liposome-forming material such as phosphatidylcholines, phosphatidylethanolamine, phosphatidylglycerols, glycerosphingolipids, and cholesterols to obtain a multilamellar vesicle or liposome. In further embodiments, the multilamellar vesicles are transformed to large unilamellar vesicles (LUVs), for example, by using a nanosizer. In one embodiment, the LUVs are administered to a patient for diagnosing and/or monitoring a disease state in which α-TTP is involved, or hepatic tocopherol and fat metabolism is involved.

### (IV) Process for the Preparation of Vitamin E Derivatives

The present disclosure also includes processes for the preparation of compounds of the Formula (I) and (II). In one embodiment, the compounds of the Formula (I) and (II), when used as multi-scale imaging agents, are synthesized using facile and quick synthetic methods in view of the short half-life of radioactive fluorine (about 109 minutes).

Accordingly, in one embodiment of the disclosure there is included a method for the preparation of the compounds of Formula (I) by electrophilic fluorination, the method comprising:

i) Contacting a Compound of the Formula (A)

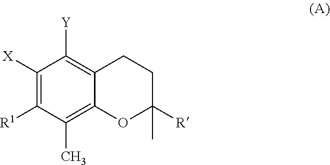

wherein
R¹ is

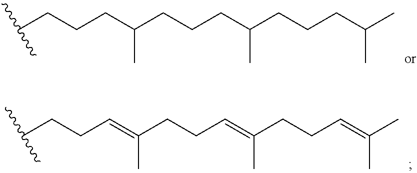 [SMILES: *CCCC(C)CCCC(C)CCCC(C)C ; *CC/C=C(\C)CC/C=C(\C)CCC=C(C)C]

R¹ is H or CH₃;
Xa is H or OH;
Ya is H or CH₃; and
wherein when X is OH, Y is H;
with a radioactive fluoro-compound for the electrophilic fluorination of the compound of the Formula (A) to obtain a compound of the Formula (I).

In one embodiment, the radioactive fluoro-compound is N-fluorobenzenesulfonimide. In one embodiment, the electrophilic fluorination reaction is conducted at a temperature of about 100° C. to about 200° C., or about 150° C. In one embodiment, the radioactive fluorine is ¹⁸F.

In another embodiment, the present disclosure also includes a second method for the preparation of the compounds of Formula (I) by electrophilic fluorination, the method comprising

i) Contacting a Compound of the Formula (B)

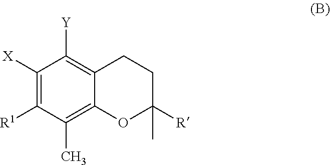

wherein
R¹ is

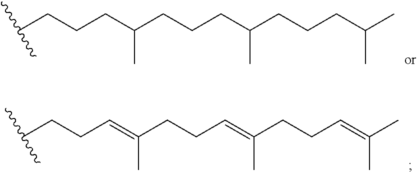 [SMILES: *CCCC(C)CCCC(C)CCCC(C)C ; *CC/C=C(\C)CC/C=C(\C)CCC=C(C)C]

R¹ is H or CH₃;
Xb is I or OH
Yb is I or CH₃;
wherein when Xb is I, Yb is CH₃, and when Yb is I, Xb is OH;
with a radioactive fluoro-compound for the electrophilic fluorination of the compound of the Formula (B) to obtain a compound of the Formula (I).

In one embodiment, the radioactive fluoro-compound is N-fluorobenzenesulfonimide. In one embodiment, the compound of Formula (B) is first reacted with a strong base such as t-butyl-lithium at a temperature of about 0° C. In another embodiment, the electrophilic fluorination is conducted at a temperature of about 0° C. In one embodiment, the radioactive fluorine is ¹⁸F.

The present disclosure also includes methods for the preparation of the compounds of Formula (I) by nucleophilic fluorination, the method comprising:

i) Contacting a Compound of the Formula (C)

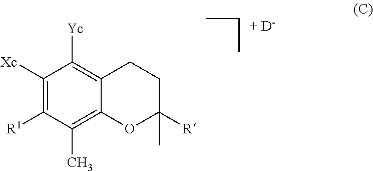

wherein
R¹ is

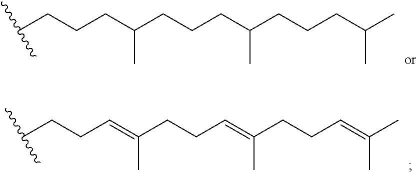 [SMILES: *CCCC(C)CCCC(C)CCCC(C)C ; *CC/C=C(\C)CC/C=C(\C)CCC=C(C)C]

R¹ is H or CH₃;
Xc is Ph-I⁺ or OH;
Yc is Ph-I⁺ or CH₃;
wherein when Xc is Ph-I⁺, Yc is CH₃, and when Yc is Ph-I⁺, Xc is OH;
and D⁻ is an acceptable anion,
with a radioactive fluoro-compound for the nucleophilic fluorination of the compound of the Formula (C) to obtain a compound of the Formula (I).

In one embodiment, the fluoro-compound for nucleophilic fluorination is radioactive tetrabutylammonium fluoride (TBAF). In one embodiment, the radioactive fluorine is ¹⁸F.

In one embodiment, the acceptable anion is halo (Cl or F), OAc, OTs, OTf, or BF₄.

In one embodiment, the nucleophilic fluorination reaction is conducted at a temperature of about 100° C. to about 200° C., or about 150° C.

Although the disclosure has been described in conjunction with specific embodiments thereof, if is evident that many alternatives, modifications and variations will be apparent to those skilled in the art. Accordingly, it is intended to embrace all such alternatives, modifications and variations that fall within the spirit and broad scope of the appended claims. In addition, citation or identification of any reference in this application shall not be construed as an admission that such reference is available as prior art to the present disclosure.

### EXAMPLES

The operation of the disclosure is illustrated by the following representative examples. As is apparent to those skilled in the art, many of the details of the examples may be changed while still practicing the disclosure described herein.

### Example 1a: Synthesis of F-Toc—Synthesis of F-Toc by Electrophilic Fluorination

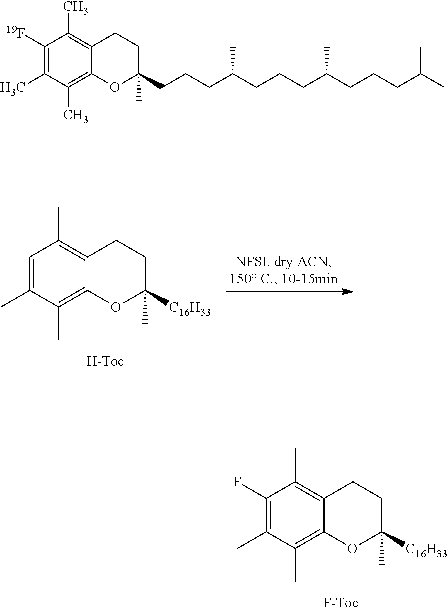

H-Toc (1 eq) was mixed with N-fluorobenzenesulfonimide (1 eq) and stirred in dry acetonitrile as a 1M solution for 10-15 min at 150° C. The reaction was cooled to room temperature, extracted with CH₂Cl₂ and water, the organic phase dried over Na₂SO₄ and evaporated down to dryness. The crude product was filtered through a silica plug with hexane to remove polar byproducts. Silica column chromatography (gradient Hexane to Hexane/CH2Cl2 99:1) afforded F-Toc (44%) as a clear oil.

### Example 1b: Synthesis of F-Toc

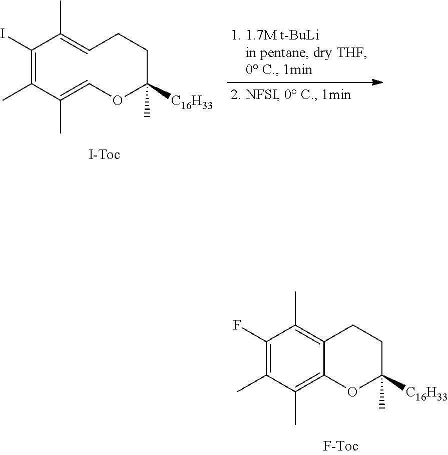

To a 0.85M solution of I-Toc (1 eq) in dry THF at 0° C. under an N₂ atmosphere was a 1.7M t-BuLi solution in pentane (2 eq) added and stirred for 1 min. An 0.35M N-fluorobenzenesulfonimide (2 eq) solution in THF was slowly added and stirred for 1 min at 0° C. The reaction was quenched with methanol, the solvents evaporated, extracted with CH₂Cl₂ and water, the organic phase dried over Na₂SO₄ and evaporated down to dryness. Silica column chromatography (gradient Hexane to Hexane/CH₂Cl_{2 99:1}) afforded F-Toc (15%) as a clear oil.

### Example 1c: Synthesis of F-Toc by Nucleophilic Fluorination

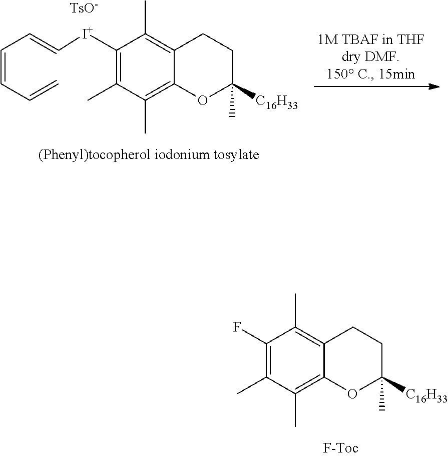

(Phenyl)tocopherol iodonium tosylate (1 eq) was dissolved in DMF as a 5 mM solution, 1M tetrabutylammonium fluoride in THF (1M TBAF in THF, 1 eq) was added and stirred for 15 min at 150° C. The solvent was evaporated and the residual mixture partitioned between hexane and water. The organic phase was dried with Na₂SO₄, filtered and purified over a small SiO₂ column with hexane. Silica column chromatography (gradient Hexane to Hexane/CH₂Cl₂ 99:1) afforded F-Toc (24%) as a clear oil.

TLC: Rf=0.27 (Hexane).

1H-NMR (400 MHz, CDCl3): δ 2.60 (t, J=6.80 Hz, 2H, ArCH2CH2), δ 2.16 (d, J=6.80 Hz, 3H, ArCH3), δ 2.12 (d J=1.60 Hz, 3H, ArCH3), δ 2.11 (s, 3H, ArCH3), δ 1.81 (enant. dt, J=6.80 Hz, 2H, ArCH₂CH₂), δ 1.65-1.04 (m, 21H, phytyl-CH/CH₂+2′R—CH3) δ 8.88 (m, 12H, phytyl-CH₃).

13C-NMR (100 MHz, CDCl3): 154.45, 152.13, 147.07, 147.06, 123.10, 123.06, 121.41, 121.22, 119.08, 118.90, 117.60, 117.56, 74.91, 39.86, 39.77, 39.38, 37.57, 37.53, 37.45, 37.40, 37.33, 37.29, 32.79, 32.69, 31.26, 31.20, 29.71, 27.99, 24.82, 24.45, 23.81, 22.73, 22.63, 21.02, 20.36, 20.34, 19.75, 19.69, 19.64, 19.60

19F-NMR (400 MHz, CDCl3): −131.49, −131.50 (d, J=4 Hz, 1F, Ar—F).

MS [EI+]: m/z 432.49 (M, 10%), m/z 205.18 (100%). HRMS Calculated for C₂₉H₅₁NO 429.3971; found: 432.3762.

### Example 2: Synthesis of (2R)-6-iodo-2,5,7,8-tetramethyl-2-(4.8.12-trimethyl-tridecyl)-chroman

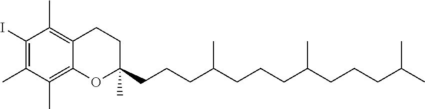 [SMILES: Cc1c(C)c2c(c(C)c1I)CC[C@@](C)(CCCC(C)CCCC(C)CCCC(C)C)O2]

I-Toc (126 mg, 0.303 mmol) and 1-iodopyridin-1-ium chloride (69 mg, 0.303 mmol) were stirred in methanol (3 ml) for 5 h at room temperature. The yellow/white emulsion was evaporated and the residue extracted with CH₂Cl₂ and water. The phases were separated and the water phase was washed 3× with CH₂Cl₂. The organic phases were combined and dried over Na₂SO₄ and dried down to dryness. Silica column chromatography (Hex to Hex/CH₂Cl₂ 10:1) afforded the compound (131 mg 78.1%) as a clear oil.

TLC: R_{f}=0.48 (Hexane)

¹H-NMR (400 MHz, CDCl₃) δ 2.70 (t, J=6.80 Hz, 2H, ArCH2CH₂), b 2.50 (s, 3H, ArCH3), b 2.45 (s, 3H, ArCH3), δ 2.22 (s, 3H, ArCH3), δ 1.80 (enant. dt, J=6.80 Hz, 2H, ArCH2CH₂), b 1.65-1.08 (m, 21H, phytyl-CH/CH₂₊₂′R—CH₃) δ 8.88 (m, 12H, phytyl-CH₃)

¹³C-NMR (400 MHz, CDCl3) 151.75, 137.93, 136.78, 123.46, 117.98, 99.48, 75.67, 39.91, 39.82, 39.38, 37.54, 37.46, 37.41, 37.37, 37.30, 32.81, 32.79, 32.69, 31.51, 31.45, 28.00, 26.97, 26.02, 24.83, 24.45, 23.81, 22.74, 22.64, 22.41, 21.04, 19.76, 19.70, 19.66, 19.60, 13.46

MS [EI+] m/z 540.42 (M, 100%), 414.48 (M, 16%), 275.04 (M, 68%)

HRMS Calculated for C₂₉H₄₉OI 540.2828; found: 540.2823.

### Example 2a: Synthesis of 1-iodopyridin-1-ium Chloride

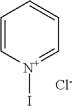

In an Erlenmeyer flask a solution of acetic acid (45 ml) and pyridine (1.49 ml, 0.0185 mol) was cooled to 0° C. and iodochloride (0.92 ml, 0.0185) was added dropwise. A yellow precipitate formed, the reaction stirred for 15 min at 0° C. and was then filtrated and washed with acetic acid (120 ml) until most of the red colour disappeared. The yellow crystals were suspended in methanol (35 ml), heated until they dissolved, filtered hot and washed with hot methanol (20 ml). The red solution was cooled for 30 min. The suspension was filtrated at room temperature and washed three times with methanol (3×20 ml). The yellow filamentous crystals were dried. The mother liquor was cooled in the fridge for 20 min. filtrated and washed with cold methanol. The second mother liquor was kept in the fridge for 2 days and filtered.

Crystal 1: 977 mg, +2: 647 mg, +3: 844 mg=55.4% of 71)

TLC: R_{f}=0.2 (CH₂Cl₂)

¹H-NMR (400 MHz, Acetone-D6) 8.84 (dd, J=6.40 Hz, J=1.60 Hz, 2H, Ar-pyridine-H), b 8.25 (tt, J=7.60 Hz, J=1.60 Hz, 1H, Ar-pyridine-H), 8.01 (d, J=6.40 Hz, J=1.20 Hz, 2H, Ar-pyridine-H)

¹³C-NMR (400 MHz, Acetone-D6) 148.61, 140.50, 127.20

MS [ESI+] m/z 205.9 (M-CI, 100%)

MS Calculated for C₄H₄NICI 240.916.

¹²³ICI can be used to prepare ¹²³I derivatives (see Dewanjee).

### Example 3—Synthesis of BODIPY Compounds

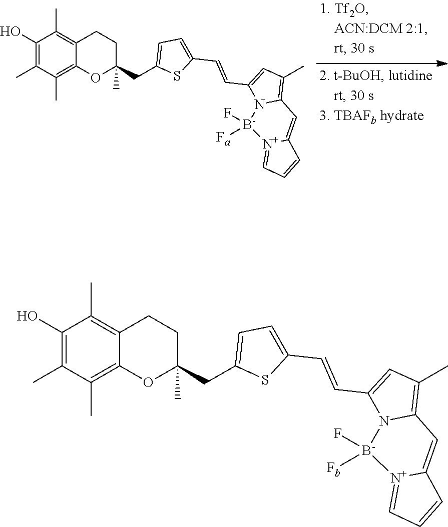

See Ghelfi, M., Ulatowski, L., Manor, D. & Atkinson, J. Synthesis and characterization of a fluorescent probe for α-tocopherol suitable for fluorescence microscopy. Bioorganic & Medicinal Chemistry 24, 2754-2761 (2016).

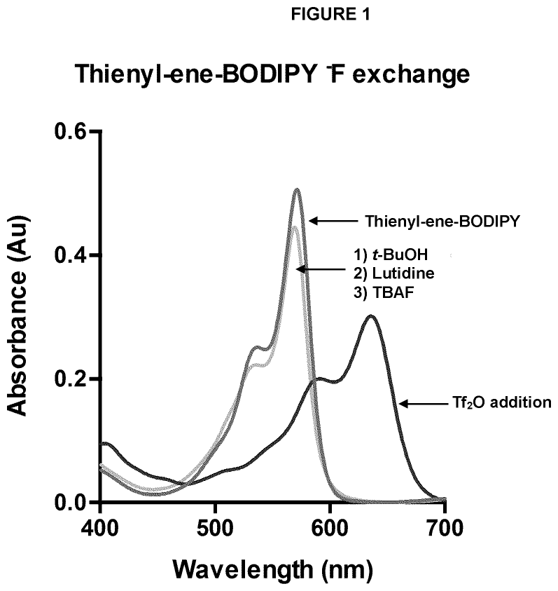

FIG. 1 shows the fluorine exchange of thienyl-ene-BODIPY, followed by UV/Vis. In a cuvette 3 (32 μM, dry ACN:DCM 2:1) was scanned from 200-700 nm. TMSOTf (4 eq) was added to the cuvette, shaken and measured. t-BuOH (4 eq), Lutidine (4 eq) and TBAF hydrate (4 eq) were added, shaken and UV/Vis absorption measured. λ max: BODIPY-F 571 nm, BODIPY-OTf 639 nm.

### Example 4: Liposome Stock Solution Preparation

Phospholipid films were prepared by transferring the desired volumes of stock solutions of POPC:POPG:Cholesterol: α-Toc (or thienyl-BODIPY tocopherol TBtoc), at a molar ratio of 7:3:4:0.1, to a glass vial. Organic solvent was then removed with an N₂ stream and gentle heating, followed by drying in vacuo (6 h). Lipid films were hydrated with phosphate buffered saline (pH 7.4) to a concentration of 10 mg/mL. The resulting multilamellar vesicle (MLV) suspensions were incubated at 30° C. and subjected to five freeze/thaw cycles. Large unilamellar vesicles (LUVs) were prepared by passing the MLV suspensions through a single use sterile 50-nm NanoSizer (T&T Scientific, Knoxville, Tenn.) 31 times at room temperature.

### Example 5: Viability of Mouse Cells Cultured in the Presence of Tocopherol Derivatives

### Methods and Materials

C2C12 mouse myoblasts and mouse embryonic fibroblasts (MEFs) were cultured in DMEM supplemented with 10% (v/v) FBS, 4500 mg/L glucose, 4 mM L-glutamine, 1 mM sodium pyruvate, 2% (v/v) MEM nonessential amino acid solution, and penicillin (50 I.U./mL)/streptomycin (50 μg/mL) solution (complete media). Cells were cultured in a humidified 5% CO2 atmosphere within a Thermo Forma Series II water-jacketed CO₂ incubator maintained at 37° C. Cells were transferred to fresh 96- or 6-well plates (2,000 cells/well & 60,000 cells/well, respectively) in the evening prior to commencing tocopherol treatments.

Each tocopherol was dissolved in sterile 100% DMSO to yield a 1 M stock solution. Less concentrated stock solutions were subsequently prepared using ten-fold serial dilutions. All tocopherol solutions were stored at −20° C. To treat cultured cells, media was replaced with complete media containing freshly-added tocopherol; the final amount of vehicle (DMSO) for all concentrations tested was 0.1% (v/v).

### MTT Tetrazolium Reduction Assay

Media was discarded, wells were washed once with phenol red-free complete culture media, and 100 μL/well phenol red-free complete culture media containing 0.45 mg/mL MTT was added. Two hours later, solubilization solution [40% (v/v) dimethylformamide, 2% (v/v) glacial acetic acid, 16% (w/v) sodium dodecyl sulfate, pH 4.7] was added (100 μL/well) and well contents were gently mixed by re-suspension to dissolve the formazan precipitate. Plates were incubated at room temperature in darkness for 2 h before recording absorbance at 570 nm using a Bio-Tek PowerWave Microplate UV-Vis spectrophotometer (Winooski, Vt., USA). For each plate, background signal averaged from cell-free wells containing vehicle treatments was subtracted

### Results and Discussion

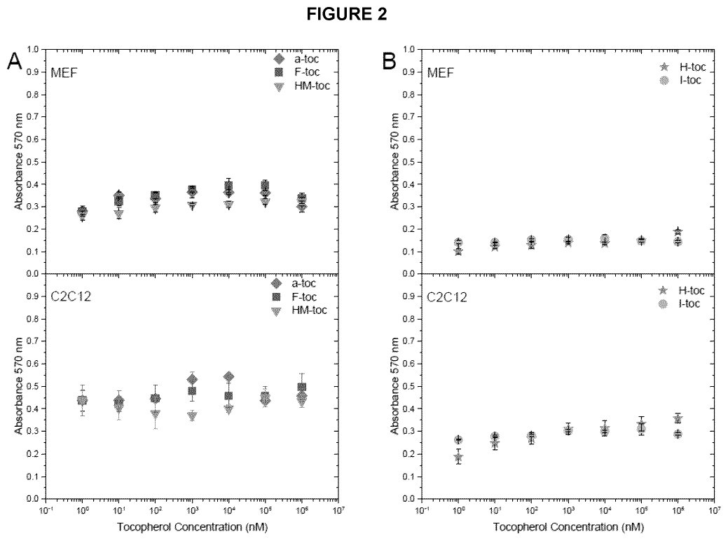

Cell death of both the C2C12 and MEFs in the presence of tocopherol derivatives at concentrations spanning five orders of magnitude was monitored. FIG. 2 clearly shows that there was no sensitivity to tocopherol, 6-fluoro-tocopherol, or the hydroxymethyl tocopherol in both the C2C12 myoblasts and the fibroblasts.

FIG. 2 shows the viability of mouse cells cultured in the presence of tocopherol derivatives at concentrations ranging from 1 nM to 1 mM. C2C12 mouse myoblasts and mouse embryonic fibroblasts (MEFs) were treated with tocopherol derivatives (A) (a-Toc, F-Toc and HM-TOC) and (B) F-toc precursor molecules (I- or H-tocopherol) for 24 h prior to assessing viability via spectrophotometric measurement of formazan (absorbance at 570 nm) produced from the live-cell-catalyzed reduction of MTT tetrazolium. There were no significant differences between absorbance values of all tocopherol groups compared to the corresponding vehicle control (0.1% DMSO; Tukey's post-hoc test). Data points represent means±SEM, with n=4 for all conditions except for F-toc in MEFs (n=3). Note: absorbance values for α-, F- and HM-toc vehicle control groups in C2C12 cells and MEFs were 0.4343±0.0299 and 0.2984±0.0154, respectively. Absorbance values for vehicle control groups for I- and H-toc in C2C12 cells and MEFs were 0.303±0.002 and 0.151±0.004, respectively.

Furthermore, data collected on the synthetic precursors, H-tocopherol and 1-tocopherol, show no negative sensitivity in either cell line. The data shows a clear increase in absorption at 570 nm for both myoblasts, suggesting a positive impact on the cell line.

Neither the F-toc nor the tocopherol based precursors show cytotoxicity up to a concentration of 1 mM. In addition, the two synthetic pathways to produce F-toc, either by electrophilic fluorination or nucleophilic fluorination, can be completed in under 15 minutes (see Methods and Materials). This expedient synthesis addresses the technical issues of optimized and expedient production of the F-18 labeled agent since F-18 has a short half-life of 109 minutes

### Example 6: Viability of Mouse Cells Cultured in the Presence of TBtoc

C2C12 mouse myoblasts and mouse embryonic fibroblasts (MEFs) were cultured in DMEM supplemented with 10% (v/v) FBS, 4500 mg/L glucose, 4 mM L-glutamine, 1 mM sodium pyruvate, 2% (v/v) MEM nonessential amino acid solution, and penicillin (50 I.U./mL)/streptomycin (50 μg/mL) solution (complete media). Cells were cultured in a humidified 5% CO₂ atmosphere within a Thermo Forma Series II water-jacketed CO₂ incubator maintained at 37° C. Cells were transferred to fresh 96- or 6-well plates (2,000 cells/well & 60,000 cells/well, respectively) in the evening prior to commencing tocopherol treatments.

Each tocopherol was dissolved in sterile 100% DMSO to yield a 1 M stock solution. Less concentrated stock solutions were subsequently prepared using ten-fold serial dilutions. All tocopherol solutions were stored at −20° C. To treat cultured cells, media was replaced with complete media containing freshly-added tocopherol; the final amount of vehicle (DMSO) for all concentrations tested was 0.1% (v/v).

### MTT Tetrazolium Reduction Assay

Media was discarded, wells were washed once with phenol red-free complete culture media, and 100 μL/well phenol red-free complete culture media containing 0.45 mg/mL MTT was added. Two hours later, solubilization solution [40% (v/v) dimethylformamide, 2% (v/v) glacial acetic acid, 16% (w/v) sodium dodecyl sulfate, pH 4.7] was added (100 μL/well) and well contents were gently mixed by re-suspension to dissolve the formazan precipitate. Plates were incubated at room temperature in darkness for 2 h before recording absorbance at 570 nm using a Bio-Tek PowerWave Microplate UV-Vis spectrophotometer (Winooski, Vt., USA). For each plate, background signal averaged from cell-free wells containing vehicle treatments was subtracted.

### Trypan Blue Exclusion Assay

Media was discarded, wells were washed once with phosphate-buffered saline, and cells were harvested via trypsinization. After centrifugation (240 g, 3 min), cell pellets were re-suspended in complete culture media and subsequently diluted in 0.4% (w/v) Trypan Blue solution. Three minutes later, the numbers of viable (non-stained) cells were counted using a hemocytometer (Hausser Scientific, Horsham, Pa.) viewed under a Hund Wetzlar Wilovert Inverted Phase-Contrast light microscope (Fisher Scientific, Mississauga, ON, Canada).

### Results and Discussion

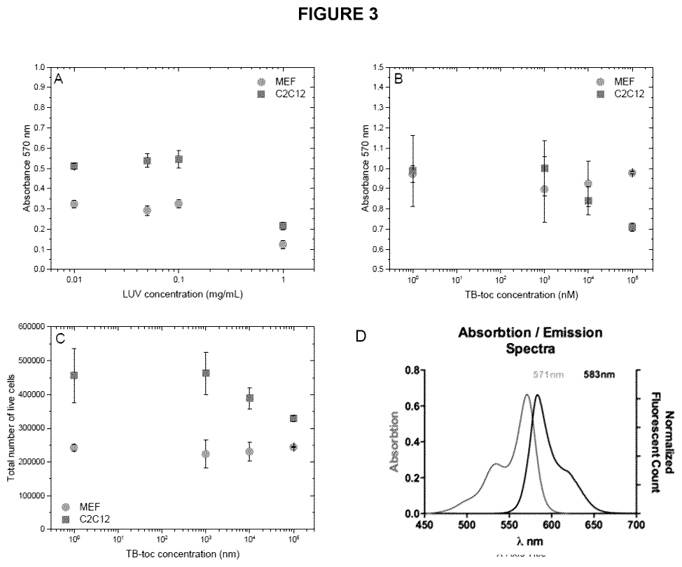

Like the other tocopherol derivatives, TB-toc was introduced to cell cultures of mouse myoblasts and fibroblasts at concentration ranging over five orders of magnitude (as shown in FIG. 3). The cultured myoblasts respond to the presence of TB-toc.

FIG. 3 shows the viability of mouse cells cultured in the presence of LUVs and BODIPY-tocopherol. C2C12 mouse myoblasts and MEFs were treated with LUVs (A) and BODIPY-tocopherol (B) prior to assessing viability via spectrophotometric measurement of formazan (absorbance at 570 nm) produced from the live-cell-catalyzed reduction of MTT tetrazolium. Data points represent means±SEM (LUV: n=8; TB-toc: n=2). (C) C2C12 and MEFs were treated with BODIPY-tocopherol for 24 h before determining the number of cells excluding Trypan blue dye. Data points represent means±SEM (n=2). The number of viable cells in the vehicle control groups for C2C12 cells and MEFs were 424,875±31,125 and 249,000±3,000 respectively. (D) TB-toc absorption and emission spectra. Data reproduced from Ghelfi (Ghelfi 2016).

### Example 7: Viability of Mouse Cells Cultured in the Presence of LUVs

Phospholipid films were prepared by transferring the desired volumes of stock solutions of POPC:POPG:Cholesterol:αToc (or TBtoc), at a molar ratio of 7:3:4:0.1, to a glass vial. Organic solvent was then removed with an N₂ stream and gentle heating, followed by drying in vacuo. Lipid films were hydrated with phosphate buffered saline (pH 7.4) to a concentration of 10 mg/mL. The resulting multilamellar vesicle (MLV) suspensions were incubated at 30° C. and subjected to five freeze/thaw cycles. Large unilamellar vesicles (LUVs) were prepared by passing the MLV suspensions through a single use sterile 50-nm NanoSizer (T&T Scientific, Knoxville, Tenn.) 31 times at room temperature.

LUVs composed of POPC:POPG:Cholesterol:αToc (7:3:4:0.1) sized by a 50-nm (diameter) pore were prepared. The effective hydrodynamic radius of LUVs prepared in this manner were measured to be 81.9 nm by dynamic light scattering. This lipid composition is already applied to the liposome-Doxorubicin suspensions (see U.S. Pat. No. 4,898,735). However, this composition lends itself to the expeditious and simple incorporation. Specifically, the incorporation of the charged POPG which makes extrusion of 50-nm easier, and avoids the presence of multi-lamellar species (Heberle 2016). We monitored cell death of both the C2C12 and MEFs in the presences LUVs concentrations spanning 3 orders of magnitude. The data demonstrate that the LUVs are non-toxic until 1 mg/mL and we can safely utilize the LUVs up to 0.1 mg/mL in both cell lines tested. Previous studies used 1 mg/mL of phospholipids (Huang1975) and 3 mM, which is approximately 2 mg/mL (Batzri1975) in similar tests with cultured Chinese hamster v79 cells and amoebae, respectively. In both cases, the concentrations examined did not appear to harm the cells.

### Example 8: TBtoc Uptake

C2C12 mouse myoblasts and mouse embryonic fibroblasts (MEFs) were cultured in DMEM supplemented with 10% (v/v) FBS, 4500 mg/L glucose, 4 mM L-glutamine, 1 mM sodium pyruvate, 2% (v/v) MEM nonessential amino acid solution, and penicillin (50 I.U./mL)/streptomycin (50 μg/mL) solution (complete media). Cells were cultured in a humidified 5% CO₂ atmosphere within a Thermo Forma Series II water-jacketed CO₂ incubator maintained at 37° C. Cells were transferred to fresh 96- or 6-well plates (2,000 cells/well & 60,000 cells/well, respectively) in the evening prior to commencing tocopherol treatments.

Fluorescence imaging of live cells was performed using Zeiss Axio Observer.Z1 inverted light/epifluorescence microscope equipped with ApoTome.2 optical sectioning, a Plan-Apochromat 63x/1.40 Oil DIC M27 objective lens, and a Hamamatsu ORCA-Flash4.0 V2 digital camera. Both the intensity of fluorescence illumination achieved via an X-Cite 120 LED light source and camera exposure times were held constant between experiments. BODIPY fluorescence was viewed using excitation and emission wavelength filter sets of 540-552 nm and 590-660 nm, respectively, with set excitation and emission wavelengths of 587 nm and 610 nm, respectively (Zeiss Item #411003-0010-000). Z-stack series consisted of approximately 20-40 slices taken at 0.32 nm intervals and were rendered into 2D maximum intensity projections using the “extended depth of focus” processing tool using Zeiss Zen 2 (blue edition) microscopy software. The microscope stage and objective were maintained at 37° C. using a TempModule S-controlled stage heater and objective heater (PeCon, Erbach, Germany). A humidified 5% CO₂ environment was achieved via tubing connected to a humidified CO₂ culture incubator. One day prior to imaging, cells were seeded onto MatTek poly-D-lysine-coated glass bottom culture dishes in complete culture media devoid of phenol red. Fluorescence intensity was analyzed from the microscope images using the ImageJ software.

The newly synthesized multi-modal imaging agent, TB-toc (Ghelfi 2016) has a peak absorption at 571 nm with a smaller peak at 530 nm and the emission spectra has a maximum fluorescence wavelength of 583 nm (Ghelfi 2016). We utilized the fluorescent properties of TB-toc to monitor its uptake into cells.

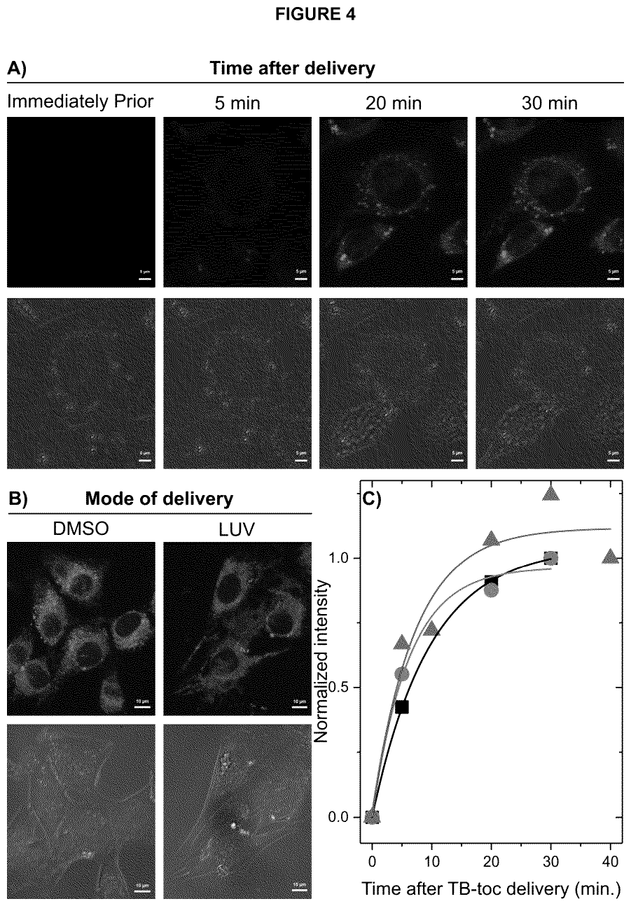

TB-toc was delivered to the cells using two methods, in DMSO stock solutions and in LUVs. TB-toc was introduced to the cultures at approximately the same concentration of our MIA. FIG. 4 illustrates the time-depended uptake of TB-toc delivered to MEFs via LUVs. The data show that TB-toc uptake is completed in approximately 30 minutes, with no observable distress to the cells. Similar results were observed for DMSO delivered TB-toc (SI), where uptake was complete in 30 minutes. However, LUV delivery yields faster initial uptake, compared to DMSO, demonstrated by the analysis of overall intensity at each time point (FIG. 4). The observation that LUV delivery yields faster initial uptake, compared to DMSO, is further visualized by the overall intensity of the images at 5 minutes after delivery (see SI). FIG. 4 shows the cellular uptake of BODIPY-tocopherol. (A) MEF uptake of TB-toc delivered with LUVs. Top row are fluorescence images (Ex. 587 nm; Em. 610 nm); the bottom row are the brightfield images. (B) Fluorescence intensity of TB-toc in C2C12 cells delivered via DMSO and LUVs after 40 min. of incubations. Images are maximum projections of z-stacks taken at 0.32 nm intervals. (C) The relative fluorescence intensity of TB-toc introduced to MEF cells via LUVs (red circles) and DMSO (black squares) and introduced to C2C12 cells via LUVs (blue triangles).

The overall intensity of the LUV delivered label is fainter than the DMSO solution (FIG. 4). However, the images suggest that delivery via DMSO and LUVs yield the same cellular distribution of TB-toc.

Targeted delivery of tocopherol and its derivatives using liposomal delivery vehicles delivers the MIA to specific cell and tissue types utilizing the plethora of existing targeted liposome systems.

### Example 9: TBtoc Characterization of Ligand Binding Ability

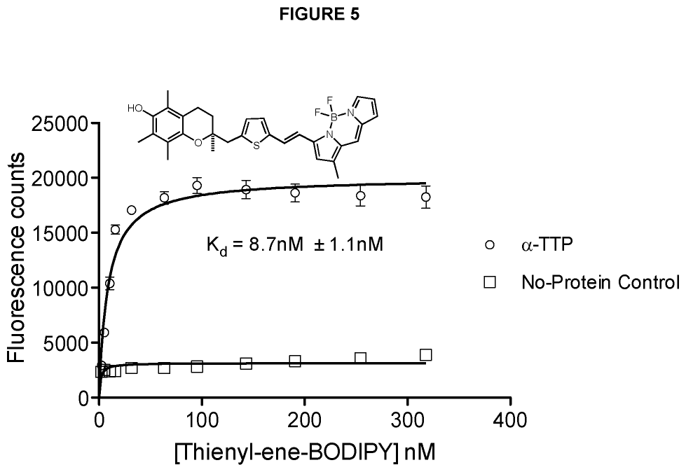

Binding assays with TBtoc and α-TTP showed reproducible saturation with dissociations constants, K_{d}, determined to be 5-10 nM when stock solutions were prepared in EtOH or dixoane (FIG. 5). FIG. 5 shows fluorescence titration of dioxane solutions of TBtoc to 0.4 μM α-TTP in TKE buffer (50 mM Tris-HCl, 100 mM KCl, 1 mM EDTA, pH 7.4) illustrating binding to α-TTP. Assays were performed in triplicate and errors bars show standard errors from the mean (SEM). The errors on the no-protein control are smaller than the symbol size used

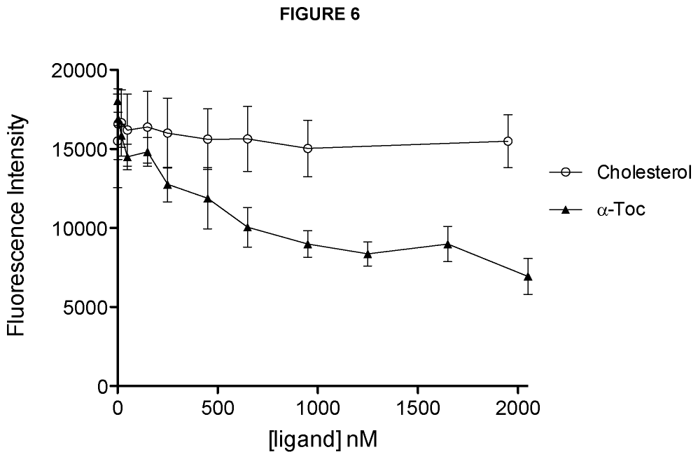

When α-TTP is pre-incubated with TBtoc, competitive assays are possible by the incremental addition of a specific ligand such as natural α-tocopherol. Cholesterol served as a negative control (FIG. 5). About 57% of the bound fluorescence could be competed of α-TTP by α-tocopherol, whereas only about 8% was lost with cholesterol. Control experiments with just the organic solvent dioxane reduced fluorescence just as much as the cholesterol solutions suggesting that the increased solvent content alone was sufficient to reduced bound ligand to a minor degree. FIG. 6 shows the competitive displacement of 0.4 μM TBtoc from a pre-incubated complex with 0.4 μM α-TTP by α-tocopherol or cholesterol (in dioxane) in TKE buffer.

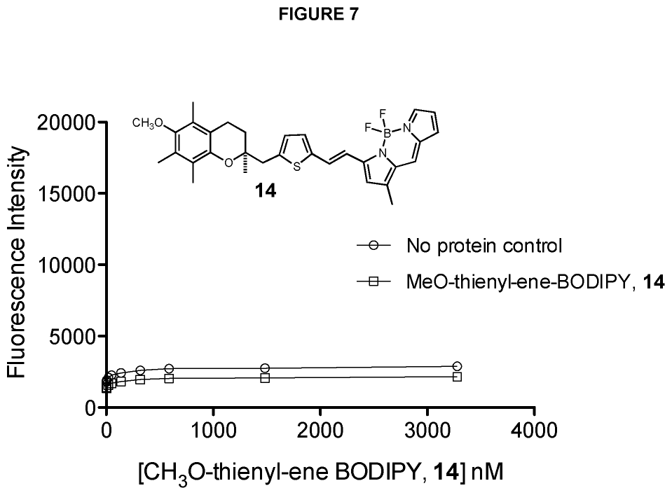

As a further control to emphasize the specificity of binding for TBtoc, the methyl ether TBtoc was prepared since it is known that the free phenol is required for specific binding to α-TTP. ^{24, 25} FIG. 7 shows that the methyl-ether of TBtoc gave no greater increase in fluorescence signal when added to a solution of α-TTP than if added to a solution containing no protein.

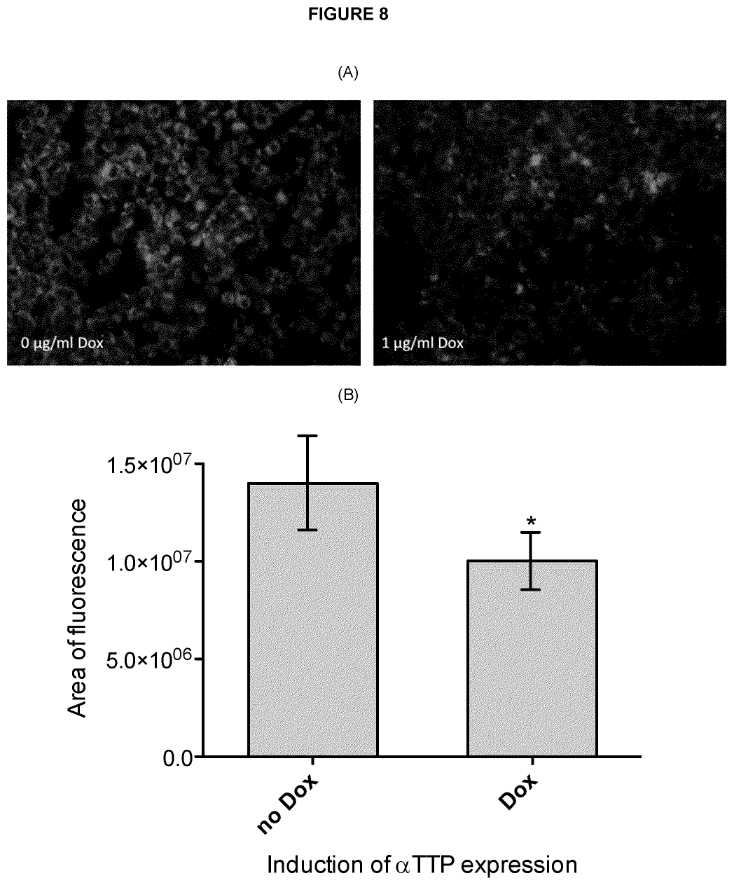

FIG. 8 shows α-TTP facilitated secretion of TBtoc. A) Representative images of McA-RH7777-TetOn-TTP cells induced to express TTP with doxycycline (1 μg/ml Dox) or treated with vehicle control (1 μg/ml Dox) and loaded with 15 μM of fetal bovine serum complexed 2 for 18 hours. After the TBtoc was removed by washing, a four hour “secretion” phase was followed by microscopic visualization of the fluorescence. B) Fluorescence was quantified in multiple fields and normalized to cell protein content. Shown are averages and standard deviations. Asterisks denote significant difference (P>0.05) between the TTP-expressing and non-expressing cells, as determined by Student's t-test.

While the present disclosure has been described with reference to what are presently considered to be the preferred examples, it is to be understood that the application is not limited to the examples described herein. To the contrary, the present disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### REFERENCES HEREIN INCORPORATED REFERENCE

S. Batzri, E. D. Korn, Interaction of phospholipid vesicles with cells Endocytosis and fusion as alternate mechanisms for the uptake of lipid-soluble and water-soluble molecules, Journal of Cell Biology, 66 (1975) 621-634.
Dewanjee, M. K. Radioiodination: theory, practice and biomedical applications Springer, DMDU 21 (1992), p. 105
A. Erhardt, W. Stahl, H. Sies, F. Lirussi, A. Donner, D. Hsussinger, Plasma levels of vitamin E and carotenoids are decreased in patients with Nonalcoholic Steatohepatitis (NASH), Eur. J. Med. Res, 16 (2011) 76-78.
Ghelfi, M., Ulatowski, L., Manor, D. & Atkinson, J. Synthesis and characterization of a fluorescent probe for α-tocopherol suitable for fluorescence microscopy. Bioorganic & Medicinal Chemistry 24, 2754-2761 (2016).
Hamza Hadi, Roberto Vettor, Marco Rossato, Vitamin E as a Treatment for Nonalcoholic Fatty Liver Disease: Reality or Myth? Antioxidants, 7 (2018) 12-13.
F. A. Heberle, D. Marquardt, M. Doktorova, B. Geier, R. F. Standaert, P. Heftberger, B. Kollmitzer, J. D. Nickels, R. A. Dick, G. W. Feigenson, J. Katsaras, E. London, G. Pabst, Subnanometer Structure of an Asymmetric Model Membrane: Interleaflet Coupling Influences Domain Properties, Langmuir, 32 (2016) 5195-5200.
P. Hirsova, S. H. Ibrabim, G. J. Gores, H. Malhi, Lipotoxic lethal and sublethal stress signaling in hepatocytes: relevance to NASH pathogenesis, J Lipid Res, 57 (2016) 1758-1770.
L. Huang, L. Huang, L. Huang, R. E. Pagano, Interaction of phospholipid vesicles with cultured mammalial cells I Characteristics of uptake, J Cell Biol, 67 (1975) 38-48.
T. Thrasher, M. F. Abdelmalek, Nonalcoholic Fatty Liver Disease, N C Med J, 77 (2016) 216-219.

1. A compound of the Formula (I) 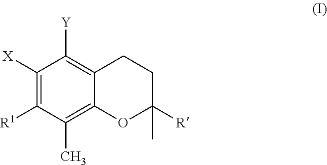
wherein
R′ is 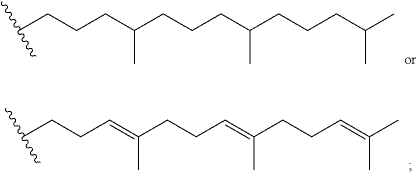 [SMILES: *CCCC(C)CCCC(C)CCCC(C)C ; *CC/C=C(\C)CC/C=C(\C)CCC=C(C)C]
R¹ is H or CH₃;
X is ¹⁸F, ¹⁹F or OH;
Y is ¹⁸F, ¹⁹F or CH₃;
wherein when X is ¹⁸F or ¹⁹F, Y is CH₃, and when Y is ¹⁸F or ¹⁹F, X is OH, and wherein one of X and Y is ¹⁸F or ¹⁹F.

2. The compound of claim 1, wherein R¹ is CH₃.

3. The compound of claim 1, wherein the compound of the Formula (I) is

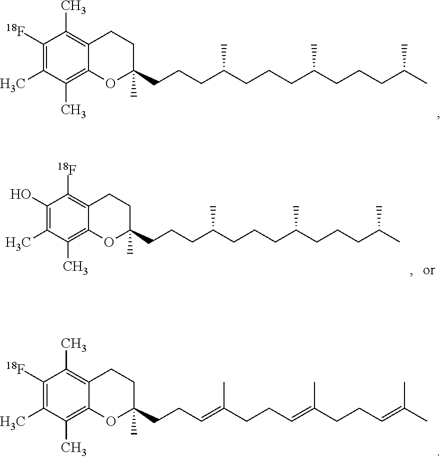

4. (canceled)

5. (canceled)

6. (canceled)

7. (canceled)

8. (canceled)

9. A liposome comprising a liposome forming material and a compound of the Formula (I) as defined in claim 1.

10. The liposome of claim 9, wherein the liposome forming material is phosphatidylcholine, phosphatidylethanolamine, phosphatidylglycerol, glycerosphingolipid, or cholesterol.

11. A method for diagnosing or monitoring a disease state involving α-TTP (α-tocopherol transfer protein), comprising:
a. administering to a patient being evaluated for the disease state an effective amount of a compound of the Formula (I) as defined in claim 1 or a liposome as defined in claim 9;
b. allowing sufficient time for the compound of Formula (I) to bind to α-TTP; and
c. diagnosing and/or monitoring the disease state using light fluorescence microscopy, and PET, MRI and/or DNP enhanced MRI.

12. The method of claim 11, wherein the disease state is non-alcoholic fatty liver (NAFL) and non-alcoholic steatohepatitis (NASH), Alzheimer's disease, Parkinon's disease, cancer, heart disease or circulatory diseases.

13. (canceled)

14. (canceled)

15. (canceled)

16. (canceled)

17. (canceled)

18. (canceled)

19. (canceled)

20. (canceled)

21. (canceled)
